(19) United States
(12) Patent Application Publication
(10) Pub. No.: US 2024/0352197 A1
(43) Pub. Date: Oct. 24, 2024

(54) DIBLOCK POLYMER

(71) Applicant: NORWEGIAN UNIVERSITY OF SCIENCE AND TECHNOLOGY (NTNU), Trondheim (NO)

(72) Inventors: Bjørn CHRISTENSEN, Trondheim (NO); Amalie SOLBERG, Trondheim (NO)

(21) Appl. No.: 18/686,773

(22) PCT Filed: Aug. 26, 2022
(86) PCT No.: PCT/EP2022/073797

§ 371 Date: Feb. 26, 2024

(30) Foreign Application Priority Data
Aug. 26, 2021 (GB) 2112232.0
Sep. 14, 2021 (GB) 2113143.8

(51) Int. Cl. C08G 81/00; B82Y 5/00; C08J 3/12; C08L 5/02; C08L 5/04

(52) U.S. Cl. CPC C08G 81/00; C08J 3/126; C08L 5/02; C08L 5/04; B82Y 5/00; C08J 2305/02; C08J 2305/04

(57) ABSTRACT

A diblock polymer comprising a first component covalently bound via a linker to a second component; wherein said first component is an oligomer comprising at least 50 mol % L-guluronic acid residues and having a degree of polymerisation n where n is at least 3; said second component is a polymer having no more than 30 mol % L-guluronic acid residues and having a degree of polymerisation m; wherein 9n>=m>=n/2.

This invention relates to nanoparticles comprising diblock polymers comprising an oligo-or polyguluronate component linked to a second polymer component, such as an oligo or polysaccharide or polyalkylene glycol. The invention further relates to the diblock polymers themselves and to uses of the nanoparticles to deliver metal ions, such as radionuclides, or organic active agents of interest to a patient. Alternatively, the diblock polymers might be used to coordinate metal ions to allow their removal from a particular environment.

### BACKGROUND OF INVENTION

Alginates are algal or bacterial polysaccharides much utilised in foods, pharmaceuticals etc. because of their mild and useful gelation properties. Most alginates have high affinities for multivalent cations like Ca ions, the binding of which leads to hydrogel formation. These phenomena are linked to the presence in alginates of sequences (blocks) of L-guluronic acid (G), which co-exist with blocks of D-mannuronic acid (M) and alternating ( . . . MG . . . ) blocks. FIG. 1 shows the structure of L-guluronic acid residues present in alginate and shows a theoretical distribution of these units with an alginate chain.

The content and distribution of G depends on the organism from which the alginate derives and is a result of the action of a family of mannuronan C5 epimerases.

Alginates may themselves be classified as block polysaccharides, the length and distribution of the three block types varying due to the inherent compositional heterogeneity of alginates. The relationship between the gelling properties of alginates with multivalent cations and the structure, sequence and chain length of alginates has been extensively investigated for decades.

It is well known how (almost) pure G blocks can be isolated from the parent alginate and separated (a standard method is partial hydrolysis combined with fractional precipitation with dilute acid: G-blocks precipitate selectively when alginate is hydrolysed at a specific pH (M- and MG blocks are soluble). In contrast, the properties of isolated M- and G-blocks and their incorporation in precisely engineered alginate-based block polysaccharides have been minimally investigated.

The present inventors have now determined that nanoparticles can be prepared from precisely engineered alginate-based block polysaccharides in which G blocks are linked in any convenient fashion to a second polymer such as an oligo or polysaccharide. The G blocks required are ones that contain a high proportion of G residues as it is these units that coordinate the metal ions or active agent and allow the spontaneous formation of nanoparticles in solution.

### SUMMARY OF INVENTION

Viewed from one aspect the invention provides a diblock polymer comprising a first component covalently bound via a linker to a second component;
wherein said first component is an oligomer comprising at least 50 mol % L-guluronic acid residues and having a degree of polymerisation n where n is at least 3;

said second component is a polymer having no more than 30 mol % L-guluronic acid residues and having a degree of polymerisation m;

wherein 9n=>m>=n/2, such as 9n=>m=>n.

For the avoidance of doubt, if n/2 is not a whole number then the value of n/2 is rounded up to the nearest whole number.

Viewed from another aspect the invention provides a diblock polymer comprising a first component covalently bound via a linker to a second component;

wherein said first component is an oligomer comprising at least 50 mol % L-guluronic acid residues and having a degree of polymerisation n where n is at least 3;

said second component is an oligo or polysaccharide having no more than 30 mol % L-guluronic acid residues and having a degree of polymerisation m;

wherein 9n=>m=>n/2 and wherein m is 20 or more if n is 20 or less.

Viewed from another aspect the invention provides a diblock polymer comprising a first component covalently bound via a linker to a second component;

wherein said first component is an oligomer comprising at least 50 mol % L-guluronic acid residues;

said second component is a second polymer having no more than 30 mol % L-guluronic acid residues;

wherein said diblock polymer forms a nanoparticle spontaneously in an aqueous solution comprising metal ions in a concentration of at least 0.1 mM of metal ions.

Viewed from another aspect the invention provides a nanoparticle comprising a diblock polymer as hereinbefore defined and positive ions, such as metal 2+ or 3+ ions or H⁺ or a charged organic compound.

Viewed from another aspect the invention provides a core shell nanoparticle comprising a diblock polymer as hereinbefore defined, said first component forming the core and said second component forming the shell of said nanoparticle,

wherein positive ions, such as metal ions and/or charged organic compounds, are ionically bound within the core of the nanoparticle.

Viewed from another aspect the invention provides a process for the preparation of a nanoparticle comprising:
(I) obtaining a guluronic acid oligomer such as by hydrolysing alginate in the presence of an acid or base to form a guluronic acid oligomer;
(II) reacting said guluronic acid oligomer with a second polymer carrying a linking group adapted to react with said guluronic acid oligomer to form a diblock polymer.
   or
(I) obtaining a guluronic acid oligomer such as by hydrolysing alginate in the presence of an acid or base to form a guluronic acid oligomer;
(II) reacting said guluronic acid oligomer with a linking group adapted to react with said guluronic acid oligomer and with a second polymer;
(III) reacting said guluronic acid oligomer with linking group with a second polymer to form a diblock polymer;
   or
(I) obtaining a guluronic acid oligomer such as by hydrolysing alginate in the presence of an acid or base to form a guluronic acid oligomer and activating said oligomer with a functional group;
(II) reacting said guluronic acid oligomer with a second polymer which is adapted to carry a functional group that reacts with the functional group of the guluronic acid oligomer to form a diblock polymer;
   and subsequently:

contacting said diblock polymer with positive ions, such as metal ions, protons or a charged organic molecule to form nanoparticles.

It is preferred if the diblock polymer formed in this process is one as previously defined herein.

It is particularly preferred if the contact between the diblock polymer and the ions is effected by dialysis or internal gelling, e.g. caused by a slow adjustment of the pH releasing a gelling ion from a suitable salt or ion complex

Viewed from another aspect the invention provides use of a nanoparticle as hereinbefore defined to deliver a metal ion or charged organic compound to a patient.

### DETAILED DESCRIPTION OF INVENTION

This invention relates to diblock polymers and their ability to form nanoparticles that coordinate a positive ion such as a metal ion or proton or a charged organic compound, such as a pharmaceutical, to allow delivery of the positive ion, e.g. metal ion or charged organic compound to a patient.

What is surprising is that by terminally attaching a second polymer such as dextran to G-alginate (=oligoguluronate=G-blocks) well-defined, highly stable, nanoparticles can be formed when positive ions such as calcium ions are contacted with the diblock polymer.

In contrast, alginates themselves (i.e. without the G-block concentration required in the present invention) tend to form hydrogels in the presence of the aqueous metal ion solution and G-blocks alone form precipitates.

Whilst the main target of the invention is nanoparticles which coordinate metal ions, the coordination of protons is also possible.

In this embodiment, another chain-chain interaction comes into play. Low pH leads to carboxylate protonation (—COO—+H+═—COOH). The pKa of alginates is around 3. Well below this value, alginates no longer coordinate metals but precipitate or form so-called ‘acid gels’. G-blocks normally precipitate (the means by which they can be isolated). The diblocks of the invention are therefore capable of forming nanoparticles at low pH, e.g. 3 or less. This formation is reversible: the nanoparticles redissolve when pH>pKa.

The invention requires the combination of a first block (or first component) which is a L-guluronic acid oligomer and a second block (or second component) which is a polymer such as an oligo or polysaccharide or polyalkylene glycol. Ideally the second polymer is water soluble. The term water soluble is used herein to define a material which has a solubility in water of at least 10 g/L at 20° C.

The second polymer should be attached terminally to the L-guluronic acid oligomer, i.e. via functionality at the end of the L-guluronic acid oligomer. It is also preferred if the second polymer is connected via a terminal position to the L-guluronic acid block. The diblock can therefore be considered “linear”, i.e. where both blocks are connected via terminal positions on each respective block.

### Guluronic Acid Oligomers

The invention requires the use of guluronic acid oligomers (G oligomer) as the first component in the diblock polymer. These oligomers are readily obtained from alginate. Native alginate chains do not contain a sufficient concentration of G residues and hence the native alginate should be subjected to hydrolysis, e.g. in acid or base, to generate guluronic acid oligomers in which the content of guluronic acid residues is higher. Guluronic acid oligomers of interest are L-guluronic acid oligomers.

The alginate from which the guluronic acid oligomers are prepared is preferably one with a high guluronic acid content. Such alginates are known. It may be that different native alginates can be used to generate guluronic acid oligomers of different degrees of polymerisation.

The use of acid hydrolysis, e.g. using a strong acid such as sulphuric or nitric acid, is preferred as a method for degrading the natural alginate chains. The hydrolysis process can be effected simply by exposing the native alginate to the acid or base. Conveniently this can be effected at room temperature but elevated temperatures can also be used. Stirring of the reaction mixture ensures fractionation occurs efficiently.

Guluronic acid oligomers of use in the invention may have a degree of polymerisation in the range of 3 to 100, such as 5 to 80, especially 10 to 50. A further preferred range is 10 to 40. In practice, it is challenging to obtain very long G blocks from alginate and hence the use of shorter blocks with a DP of 32 to 50 is preferred.

The degree of polymerisation can be determined via NMR and represents the number of all monomer residues within the oligomer. As noted below, not all these monomer residues are guluronates but at least 50% of them must be guluronate residues.

The degree of polymerisation can be controlled via the length of the hydrolysis step and by the nature of the native alginate on which the hydrolysis is effected. Longer hydrolysis reaction leads to lower degrees of polymerisation and vice versa. For the avoidance of doubt DP=degree of polymerization=number of monomers per chain. For example, a polymer GGGGG, GGGGM, or MGMGM have a DP=5 (i.e. n=5).

The degree of polymerisation of the guluronic acid oligomer is generally chosen depending on the nature of the positive ion being coordinated and on the nature of the second copolymer. If the degree of polymerisation of the guluronic acid oligomer is low then to ensure the formation of nanoparticles, the second polymer tends to have a higher degree of polymerisation (DP). In general, if the metal ion being coordinated is large (e.g. Ba) then lower degrees of polymerisation might be employed than if the metal ion is smaller, e.g. Ca.

Alternatively viewed, the weight average molecular weight (Mw) of the guluronic acid oligomers may be in the range of 1000 to 40,000. Mw can be determined using GPC, light scattering, or a combination of both.

It will be appreciated that guluronic acid oligomers may be prepared from alginate by methods known in the art including hydrolysis, enzymic degradation (e.g. using lyases), or alkaline beta-elimination. The skilled person can devise suitable methods for forming these oligomers. Guluronic acid oligomers may contain some other monomer residues however it is essential that the guluronic acid content in the guluronic acid oligomers is at least 50 mol %, preferably at least 70 mol %, especially at least 85 mol %. The idea is to prepare guluronic acid oligomers in which the guluronic acid concentration is much higher than in the native alginate. The alginate is fractionated and oligomers which are lower in guluronic acid are removed. Only the oligomeric blocks with high G content are interesting. High G content improves the metal ion binding selectivity.

Alternatively viewed, the guluronic acid oligomer is one in which 50% or more of the monomer residues are L-guluronic, preferably 70% or more such as 85% or more of the monomer residues. The F_{G} value therefore is 0.5 or more, such as 0.7 or more, especially 0.85 or more. The use of pure guluronic acid oligomers is, of course, possible (e.g. 99 mol % or more of an F_{G} of 0.99). Other residues that might be present in the guluronic acid oligomers present include mannuronate.

The hydrolysis reaction leads to break up of the polymer chains and the target guluronic acid oligomers can be fractionated from the mix of oligomers that form.

It will be appreciated that a mixture of guluronic acid oligomers might be used when preparing the diblock polymers of the invention. Once the native alginate is hydrolysed and the high G content oligomers are isolated, such a mixture might be used as the first component in the diblock polymers of the invention or further purification might be used to isolate a single oligomer or a mixture containing fewer different oligomers. The skilled person can tailor the nature of the guluronic acid oligomer first component depending on the required properties of the nanoparticles. What is required however is that the mixture contains oligomers in which substantially all the components have at least 50 mol % guluronic acid residues.

Determining the number of repeating units within the guluronic acid oligomer and determining the number of guluronic residues within the guluronic acid oligomer can be achieved using known analytical techniques such as NMR. MALS, SEC-MALS and viscometry can also be used to determine the Mw of a polymer and that information can also be used to determining the number of repeating units or monomers within a polymer.

The guluronic acid oligomers must then be linked to the second polymer via any convenient chemistry. The nature of the hydrolysis of the alginate means that the guluronic acid oligomers contain a carbonyl group, such as aldehyde functionality. This carbonyl, or specifically aldehyde, functionality can be exploited when joining the guluronic acid oligomers to the second polymer. This carbonyl functionality is preferably positioned at the end of the guluronic acid oligomer.

### Linker

The guluronic acid oligomers are joined to the second polymer via a linker. The nature of the linker is not crucial and the skilled chemist can devise many ways of joining a guluronic acid oligomer to a second polymer. In theory this linker could simply be one atom that allows the two components of the diblock polymer to be linked, e.g. an —O— atom. Preferably however a dedicated linking molecule is used.

Any suitable covalent chemistry might be used with suitable functionalisation of reactants to create appropriate nucleophiles and electrophiles. The use of click chemistry is a particularly preferred method for joining the larger molecules. For example, an aminooxy-azide is readily reacted with an aminooxy-DBCO in a well-known click chemistry reaction. Functionalisation of the reactants with complementary click groups allows simple connection of the reactants. The linker in this embodiment therefore becomes the atoms between the L-guluronic acid oligomer and the second polymer. A preferred linker may therefore include a triazole group (formed by the click reaction of the alkyne and azide).

The linker of the invention is preferably multifunctional, such as difunctional or trifunctional. In one embodiment, a single linker is used that is difunctional, i.e. it must be capable of reacting with both reactants. The linking of the two components can be effected simultaneously but more conveniently one of the component is first reacted with the linker and subsequently the other component is reacted with the functionalised component.

Ideally, the linker is a small molecule with an Mw of less than 300 g/mol, such as 50 to 200 g/mol. It is however, possible to use larger linking groups such as a polyalkylene oxide chain. Preferably such a polymeric linker will have fewer than 20 repeating units.

Conveniently, the linking reaction will exploit terminal masked carbonyl/aldehyde groups in the guluronic acid oligomers and second polymer, if present. Ideally therefore, the linking reaction involves a reductive amination, amination or reaction involving click chemistry, e.g. with a functional group selected from azide, alkyne, thiol, alkene etc. The use of a dioxyamine or a dihydrazide is preferred.

The linker may therefore form a Schiff base (oxime or hydrazone) with the first or second components. Conveniently, one of the components is functionalised with a difunctional reductive amination type reagent, such as a O,O″-1,3,-propanediylbishydroxylamine dihydrochloride or adipic acid dihydrazide (ADH).

The other component is then combined to link the two blocks. Details are provided in the experimental section below and can be readily adapted by the skilled chemist.

Conveniently, the linker is a difunctional linker in which there are terminal functional groups linked by an alkylene chain, such as a C₁₋₁₀ linear alkylene chain. Functional groups of interest include O—NH₂ or —CO—NH—NH₂. Longer linkers might change the viscosity of the diblock polymer so linker length is a further tool that the skilled chemist can use to change the properties of the diblock polymer.

Once the reaction is complete, the Schiff bases might be reduced, e.g. to form a stable amine). Suitable reducing agents include picoline borane or sodium cyanoborohydride. Such a species might be chemically more stable than an oxime or hydrazone.

In FIG. 2, there is a description of the reaction of guluronate with PDHA or ADH to form the oxime or hydrazone with subsequent reduction to the N-oxide or hydrazine. It will be appreciated that the hydrazone has an equivalent form-a pyranoside. There might also be equivalent cyclic forms, such as furanosides.

Ideally, the linker should link terminal positions of the guluronic acid oligomer and the second polymer.

The skilled person will be readily able to devise suitable chemistry to link the two components. In one embodiment, the linker might contain 5 to 20 backbone atoms (i.e. the chain linking the two blocks is 5 to 20 atoms in length). For example, a O—CH₂—CH₂—CH₂—CH₂—O linker contains 6 backbone atoms.

In some embodiments, the linker may comprise a short chain polyalkylene glycol, such as a PEG. Such a chain may have up to 10 repeating units, e.g. up to 5 such units.

### Second Polymer

The second component in the diblock polymer is a polymer such as an oligo or polysaccharide, poly(meth)acrylate or polyalkylene glycol. It will be appreciated that the second soluble polymer must be different from the guluronic acid oligomer. The second polymer does not therefore contain more than 30 mol % guluronic acid residues. Ideally, it does not contain any guluronic acid residues. The second polymer is preferably not one that derives from alginate.

Alternatively viewed the second polymer is one that does not interact with the cation coordination the G-blocks

It is preferred if the second polymer is a water soluble polymer. Some insoluble polymers may also be used, especially those with a low degree of polymerisation, such as insoluble chitin oligomers with a DP of 6 to 40.

The second polymer is one that, when linked to the G-oligomer, forms a nanoparticle in the presence of positive ions such as metal ions. Second polymers that form a precipitate in those circumstances are excluded.

It is preferred if the second polymer has a higher weight average molecular weight (Mw) than the guluronic acid oligomer. Ideally, the second polymer has a Mw at least 2 times that of the guluronic acid oligomer, such as 3 to 8 times higher. If the second polymer has a Mw which is too high however (e.g. 20× or more the Mw of the guluronic acid oligomer) then it is more likely that a precipitate forms rather than the target nanoparticle.

Alternatively viewed, the degree of polymerisation of the second polymer should be the same as or higher than that of the guluronic acid monomer. The ratio of n to m is therefore important where n is the DP of the guluronic acid and m is the DP of the second polymer. The ratio is ideally 2:1 (nm) to 1:9 (n:m), such as 1:1 (n:m) to 1:9 (n:m). A particularly preferred ratio is 4n=>m>=n.

In general therefore, if the DP of G is much larger than the DP of the second polymer then precipitation occurs. If both oligomers are short, e.g. the DP is less than 15 for both oligomers then if the DP of G is the same as the DP of the second polymer then precipitation occurs rather than the formation of NP. If therefore the DP of the G oligomer is in the range of n=3 to 15 then the DP of the second polymer m is preferably 30 to 180.

For example, G₁₀-linker-Dex₄₀ results in the formation of nanoparticles whereas G₁₀-linker-Dex₁₀₀ precipitates (with Ca ions).

G₄₀-linker-Dex₄₀ forms nanoparticles as does G₄₀-linker-Dex₁₀₀.

If the value of m exceeds 180 then there is a risk that the diblock polymer is water soluble and hence m is preferably 180 or less.

The exact values of m and n which lead to precipitation or nanoparticles may vary depending on the nature of the positive ion being coordinated within the nanoparticle.

Without wishing to be limited by theory, it is believed that the appropriate Mw of DP of the second polymer encourages the spontaneous formation of nanoparticles in an appropriate medium, typically an aqueous medium.

The Mw of the water soluble polymer may also be less than the guluronic acid oligomer if both polymers have at least 20 repeating units.

Determining the number of repeating units within the second polymer can be achieved using well known analytical techniques such as NMR. MALS, SEC-MALS or viscometry can also be used to determine the Mw of a polymer and that information can also be used to determining the number of repeating units (monomers) within a polymer. Many commercial polysaccharides are sold with a specified degree of polymerisation.

It can be considered in fact that the water soluble polymer forms a shell where the guluronic acid oligomer forms the core of a core shell nanoparticle. The nanoparticles can be regarded as micelles or polymersomes therefore.

A preferred water soluble polymer is polyethylene glycol or an oligo or polysaccharide, especially hyaluronan, pullulan, β-1,3-glucan, heparin, glycosaminoglycans, amylose, chitosan or dextran. Dextrans are branched poly-α-D-glucosides of microbial origin having glycosidic bonds predominantly C-1→C-6″. Dextran chains are of varying lengths.

The water soluble polymer can be functionalised to carry a linker as hereinbefore described and a linking reaction between the guluronic acid oligomer and water soluble polymer can then be effected.

If the second component is a polyalkylene glycol ideally it contains at least 10 repeating units.

In a highly preferred embodiment, the guluronic acid oligomer is linked to a dextran, ideally via reductive amination, i.e. the linker comprises an N-oxide or hydrazine.

### Diblock Polymers

Engineered diblock polymers of the invention therefore comprise, such as consist of, two or more different blocks linked through a suitable conjugation method. Diblock polymers of the invention may be linear.

Diblock polymers of the invention can be named Gₙ-L-xxx herein where G is the guluronic oligomer with degree of polymerisation n. L is the linker and xxx is the second polymer, such as dextran. In particular, the diblock polymer is Gₙ-L-Dexₘ where Dex is dextran and m is the degree of polymerisation of the dextran.

The value of n is preferably 8 to 70. The value of m is preferably 30 to 180, such as 30 to 150. Ideally m is at least 2n.

The ratio of n to m is also important. The ratio is ideally 2:1 to 1:9. It is preferred therefore that 9n>m>n/2. A particularly preferred ratio is 4n=>m>=n.

### Nanoparticles

The diblock polymers of the invention self-assemble under defined conditions where one of the blocks can develop short-range attractive interactions while the other ones develop long-range repulsive interactions. Self-assembly is a spontaneous process leading to a great diversity of structures whose characteristics depends on the molecular parameters of the starting block polymers. The diblock polymers are preferably dissolved in water. On the addition of metal ions, nanoparticles form. Without being limited by theory, it is envisaged that the presence of metal ions initially allows the formation of dimers of the diblock polymers. The formation of these dimers leads, in turn to the formation of nanoparticles.

In contrast, if a diblock polymer based on two oligoguronates is used, the addition of metal ions causes the formation of solid precipitates rather than nanoparticles.

Normally an excess of metal ions are added to ensure nanoparticle formation. The concentration of metal ions required in solution varies depending on the nature of the metal ion. It will also be appreciated that a mixture of metal ions might be used. In general, the concentration of metal (2+) ions required in solution follows the order: Mg>>Mn>Ca>Sr>Ba>Cu>Pb. In some embodiments, a saturated solution might be used.

The addition of metal ions to an aqueous solution of the diblock polymer allows the spontaneous formation of the nanoparticles of the invention. Ideally, addition of the metal ions occurs using dialysis or internal gelation.

Internal gelation is a process where metal ions such as Ca is first distributed in the alginate, for example as metal carbonate microparticles, or as soluble metal complex, such as metal-EGTA or metal-EDTA complexes. A pH adjuster such as GDL is used to slowly lower pH sufficient to release metal ions from the source to induce metal-alginate gelation. In the presence of the diblocks of the invention this surprisingly gives stable nanoparticles. If an alginate is used, a hydrogel forms.

Conveniently, dialysis involves a diblock solution dialysed against a metal ion solution such as a solution of Ca ions, e.g. CaCl₂. The length of the dialysis can vary depending on the molecular weight of the diblock polymer and the pore size of the dialysis membrane. Larger polymers tend to require shorter dialysis times than smaller diblock polymers.

Typical solutions of both the diblock and the metal ion solution might be 1 to 100 mM in concentration. A buffer may also be used, such as sodium acetate.

Nanoparticles can be allowed to form for a prolonged period until a steady state is reached. That could take up to two weeks.

Alternatively, the nanoparticles might be formed by supplying a homogeneous metal ion source, such as a solution of metal ions, in a process colloquially known as “internal gelation”. The diblock polymers can be dissolved in a saline solution and subsequently contacted with a metal ion complex, e.g. CaEGTA (ethylene glycol-bis (B-aminoethyl ether)-N,N,N′,N′-tetraacetic acid). Nanoparticles are formed due to the homogeneous release of calcium ions from e.g. CaEGTA by a slow change in pH induced, for example, by the introduction of GDL (gluconodelta lactone).

Oligoguluronate-L-dextran diblocks form well-defined core-shell micelle-like nanoparticles by the introduction of calcium ions, e.g. by dialysis. The core shell particles have a strict phase separation between the G-based core and the dextran corona.

In contrast, free oligoguluronate chains precipitated under the same conditions. This is probably the first report of a stimuli-sensitive diblock polysaccharide without involving lateral modifications.

Alginates, G-blocks, and Gn-b-Xm diblocks therefore react differently with calcium salts or dilute acids: Alginates generally form macroscopic hydrogels, G-blocks precipitate out of solution, whereas Gn-b-Xm diblocks form stable nanoparticles with a core/shell structure. The alternative nomenclature Gn-b-Xm is used herein to define a deblock with Gn (G block), b as a linker and Xm as the second component.

Metal ions which can be coordinated are preferably multivalent, preferably trivalent or especially divalent. The use of group II metal ions, especially Ca, Ra, Sr and Ba ions is preferred. Other metals of interest include actinides and lanthanides such as yttrium, terbium, lutetium and actinium or some transition metals such as Cu and Zr. Cu-64 and Cu-67 are interesting alternatives for example along with terbium 149/152/155/161. In particular, radionuclides can be coordinated in the nanoparticles of the invention. Suitable radionuclides include those of actinium, thorium, radium, lutetium, gallium, technetium, bismuth, palladium, lead, samarium, iridium, astatine, rhenium, erbium, zirconium and indium.

Specific radionuclides include actinium-225, thorium-227, radium-223/224, lutetium-177 gallium-68, technetium-99, Bismuth-213, gallium-67/68, Samarium-153, Astatine-211, Rhenium-186/188, erbium-169, zirconium-89, palladium-103, iridium-192 and lead-212 and indium-111. Radioactive ions that target cancer are of particular interest.

In the case of alginates, the strong and specific interactions of G-blocks with Ca, Ra, Sr and Ba ions could be balanced by steric (repulsive) interactions brought by a neutral polymer block such as dextran conjugated to the G block.

Nanoparticles preferably have a diameter of 10 to 100 nm, such as 20 to 80 nm.

The nanoparticles can therefore be used to administer radionuclides or other interesting metal ions to a patient. They are also a convenient vehicle to store radionuclides. The nanoparticles of the invention are stable under physiological conditions, e.g. at body temperature and pH. They are injectable.

The preparation of nanoparticles comprising certain metal ions is challenging. For example, forming nanoparticles using magnesium ions is challenging as these do not combine with the diblock polymer spontaneously to form nanoparticles. Nevertheless, it would be useful if magnesium containing nanoparticles could be formed as such nanoparticles might have a higher affinity for certain targets.

It has been found that magnesium ions can be introduced into a nanoparticle via displacement of the metal ion already present in the nanoparticles. Nanoparticles can therefore be formed using, for example, calcium ions following protocols described herein and subsequently these nanoparticles are exposed to magnesium ion solutions, e.g. dialysed with such solutions. Moreover, the strength of the magnesium ion solution can be varied to change the amount of metal ions that are displaced. By increasing the concentration of magnesium ions in the solution, more metal ions are displaced from the nanoparticles. Our experiments suggest that there is an optimum concentration above which displacement becomes less effective. The skilled person can readily determine the required concentration to maximise displacement. Typically concentrations are 0.05 to 20 mM. Counterions such as halides, nitrates etc are suitable for the metal ion solutions. The inventors demonstrate that 50 to 95% of the metal ions can be displaced thus resulting in 50 to 95% displacement ions, e.g. Mg ions in the nanoparticles.

It will be appreciated that the principles of displacement could be used on a variety of different metal ion combinations to allow the introduction of different metal ions into the nanoparticles. The introduction of alkali metal ions such as sodium or potassium ions might be considered for example

In one embodiment therefore the process of the invention further comprises a step in which nanoparticles comprising first metal ions are combined with a solution of second metal ions, e.g. nanoparticles comprising calcium ions are combined with a solution of magnesium ions, so as to displace at least a portion first metal ions and replace them with a portion of second metal ions.

In a further embodiment, the nanoparticles of the invention may coordinate a charged organic molecule of biological interest such as a charged pharmaceutical. The guluronic acid core is typically negatively charged and hence it readily coordinates metal ions. The same ionic interactions would also be suitable for coordinating charged organic molecules, such as positively charged organic molecules. Many pharmaceuticals in salt form are charged and are therefore suitable for coordination in the nanoparticles of the invention. Such molecules may be used instead of or as well as metal ions.

The strength of the binding to the charged species can also be tailored depending on the G content in the first component. Higher G content tends to lead to stronger binding. Where physiological release of the charged species is important, the G content of the first component can therefore be reduced to encourage release.

In a further embodiment, the diblock polymers and hence the nanoparticles might be further functionalised to carry biological targeting compounds such as antibodies, ligands etc. This could occur before or after nanoparticle formation. It may be that these biological targeting molecules themselves carry an interesting drug. For example, a radionuclide could be coordinated to an antibody which is bound to the diblock polymers of the invention.

In one embodiment, it is envisaged that nanoparticles can be formed which include biological targeting compounds such as peptides by incorporating these biological targeting compounds into a diblock polymer that becomes part of the nanoparticle during its formation. Alternatively, a relevant biological targeting moiety might be combined with a G-block polymer that becomes part of the nanoparticle during its formation. For example, a diblock polymer comprising a G block as herein defined and a peptide can be combined with a diblock polymer of the invention, e.g. one comprising Gn-b-dextran and become incorporated into the nanoparticle as it forms.

Hence, nanoparticles containing a peptide ligand can be prepared by adding Gₙ-b-peptide to a diblock polymer of the invention, e.g. Gn-b-dextran. The ratio in this process can be used to adjust the concentration of the biological molecule in the nanoparticle.

Whilst we exemplify this concept below using a peptide, any suitable biological molecule could be used and be bound to the G-block. For example, a targeting ligand could be combined with the guluronic acid oligomer. Examples includes folates which could be activated with click chemistry linkers for binding to an azide carrying G block.

Other biological molecule include antibodies, antibody fragments, nanobodies, affibodies, peptides (such as bombesin, octreotide or RGD), peptidomimetics, aptamers (nucleic acid), small molecules (such as tyrosine receptor inhibitors), hyaluronic acid and other ligands targeting receptors or cell surface molecules overexpressed in cells representing diseased tissue.

It is envisaged that the G block bound biological moiety can be combined with the diblock polymers of the invention and spontaneously incorporated as a part of the nanoparticle that forms in the presence metal ions.

Viewed from another aspect the invention provides a process for the preparation of a nanoparticle comprising:
(I) obtaining a guluronic acid oligomer such as by hydrolysing alginate in the presence of an acid or base to form a guluronic acid oligomer;
(II) reacting said guluronic acid oligomer with a second polymer carrying a linking group adapted to react with said guluronic acid oligomer to form a diblock polymer.
   or
(I) obtaining a guluronic acid oligomer such as by hydrolysing alginate in the presence of an acid or base to form a guluronic acid oligomer;
(II) reacting said guluronic acid oligomer with a linking group adapted to react with said guluronic acid oligomer and with a second polymer;
(III) reacting said guluronic acid oligomer with linking group with a second polymer to form a diblock polymer;
   or
(I) obtaining a guluronic acid oligomer, such as by hydrolysing alginate in the presence of an acid or base to form a guluronic acid oligomer, and activating said oligomer with a functional group;
(II) reacting said guluronic acid oligomer with a second polymer which is adapted to carry a functional group that reacts with the functional group of the guluronic acid oligomer to form a diblock polymer;
   and subsequently:

contacting said diblock polymer with first positive ions, such as metal ions, protons or a charged organic compound to form nanoparticles;

contacting said nanoparticles with second positive ions such as metal ions different from those used in the previous step so that said second positive ions at least partially displace said first positive ions in said nanoparticles.

Viewed from another aspect, the invention provides a process for the preparation of a nanoparticle comprising:
(I) obtaining a guluronic acid oligomer such as by hydrolysing alginate in the presence of an acid or base to form a guluronic acid oligomer;
(II) reacting said guluronic acid oligomer with a second polymer carrying a linking group adapted to react with said guluronic acid oligomer to form a diblock polymer.
   or
(I) obtaining a guluronic acid oligomer such as by hydrolysing alginate in the presence of an acid or base to form a guluronic acid oligomer;
(II) reacting said guluronic acid oligomer with a linking group adapted to react with said guluronic acid oligomer and with a second polymer;
(III) reacting said guluronic acid oligomer with linking group with a second polymer to form a diblock polymer;
   or
(I) obtaining a guluronic acid oligomer, such as by hydrolysing alginate in the presence of an acid or base to form a guluronic acid oligomer, and activating said oligomer with a functional group;
(II) reacting said guluronic acid oligomer with a second polymer which is adapted to carry a functional group that reacts with the functional group of the guluronic acid oligomer to form a diblock polymer;
and subsequently:

contacting said diblock polymer with positive ions, such as metal ions, protons or a charged organic compound to form nanoparticles in the presence of a diblock polymer comprising a guluronic acid oligomer linked to a peptide.

### BRIEF DESCRIPTION OF THE FIGURES

FIG. 1 is a schematic representation of the biosynthesis of functional alginate, partial depolymerization and isolation of pure guluronate blocks (Gn) then terminal conjugation to an activated polysaccharide. FIG. 1 also shows the subsequent dimerization with Ca++ and Gₙ-L-Dexₘ to form particles. The formation of these dimers leads, in turn to the formation of nanoparticles.

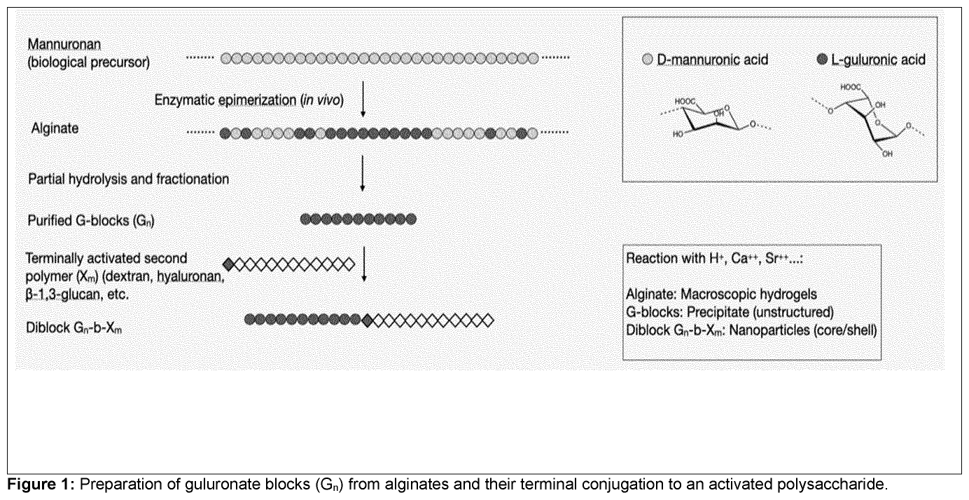

FIG. 2 shows the reaction of guluronate with PDHA or ADH and subsequent reduction using PB.

FIG. 3 shows the NMR and chemical structure of Dex₁₀-PDHA=G₃ where n represents the reduced N-oxide. The figure shows the conjugaton prior to reduction with the Schiff base. ¹H-NMR spectra of the equilibrium reaction mixture with G₃ and PDHA-Dex₁₀ is taken 500 mM AcOH[d₄] pD 4. Resonances from (E)/(Z)-oximes of the conjugate are annotated. The structure of the conjugated Gₙ=b-Dexₘ is included (=refers to unreduced oxime). ¹H-NMR spectra of purified Dex₁₀-PDHA is included for comparison.

FIG. 4 is a theoretical depiction of core shell nanoparticles of the invention with radionuclides coordinated in the core or via antibodies attached to the shell.

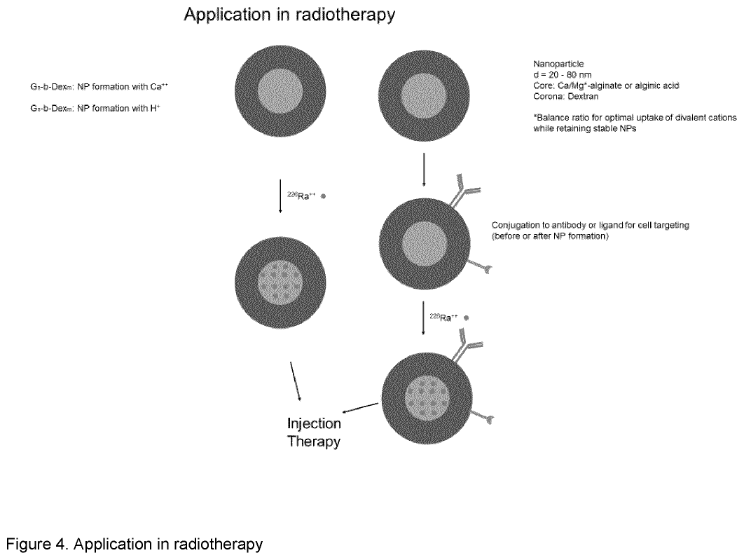

FIG. 5 shows G₁₂-PDHA-Dex₁₀₀ diblock polymer data. Residual (unreacted) G₁₂ was selectively removed by SEC (FIG. 5a). SEC-MALLS data for the diblock showed a clear shift in elution profile compared to the free blocks (FIG. 5 b).

FIG. 6 shows that nanoparticles made by G₂₄-b-Dex₃₆ remain stable (have the same particle sizes) after various treatments.

FIG. 7 shows G₂₄-b-Dex₃₆ nanoparticle sizes as a function of pH.

### EXAMPLES

### Test Methods

### SEC-MALS

The molecular weight and intrinsic viscosity of the block polymers (Gₙ-b-Gₙ and Gₙ-b-Dexₘ) was analysed by Size Exclusion Chromatograph (SEC) with Multiangle Light Scattering (MALS). Samples were dissolved in the mobile phase (0.15 M NaNO₃ with 10 mM EDTA) and filtered (0.45 μm) prior to injection. Standards were prepared using the same procedure. An Agilent Technologies 1260 IsoPump with a 1260 HiP degasser was used to maintain a flow of 0.5 ml/min during analyses. Samples (0.7-1 ml) were injected (50-100 μL per injection volume) by an Agiel Technologies Vialsampler. TKS Gel columns 4000 and 2500 were connected in series. DAWN Heleos-II and ViscoStar II detectors from Wyatt Technology were connected in series with a Shodex refractive index detector (RI-5011). Astra 7.3.0 software was used for data collection and processing.

### Preparation of Guluronic Acid Oligomers

Guluronic acid oligomers (G oligomer) with different molecular weights and degrees of polymerisation were prepared from extensively hydrolyzed, high guluronate alginate, by acid precipitation to give oligomers with various DPₙ. DPₙ was determined by NMR.

The following Guluronic acid oligomers are prepared:

DP 21, F_{G} 0.90 (where DPₙ is the average degree of polymerization and F_{G} is the fraction of monomers that are guluronic acid, i.e. the mol % of guluronic acid).
DP4. The F_{G} of the sample was >0.9.
DP10. The F_{G} of the sample was >0.9.
DP11. The F_{G} of the sample was >0.9.
DP12. The F_{G} of the sample was >0.9.

The guluronic acid oligomers are then activated to form conjugates or combined with activated dextran components to form a diblock polymer.

Adipic acid dihydrazide (ADH), O,O″-1,3,-propanediylbishydroxylamine dihydrochloride (PDHA) and 2-methylpyridine borane complex (α-picoline borane-PB) was purchased from Sigma-Aldrich.

### Preparation of Guluronate Conjugates—General Protocol

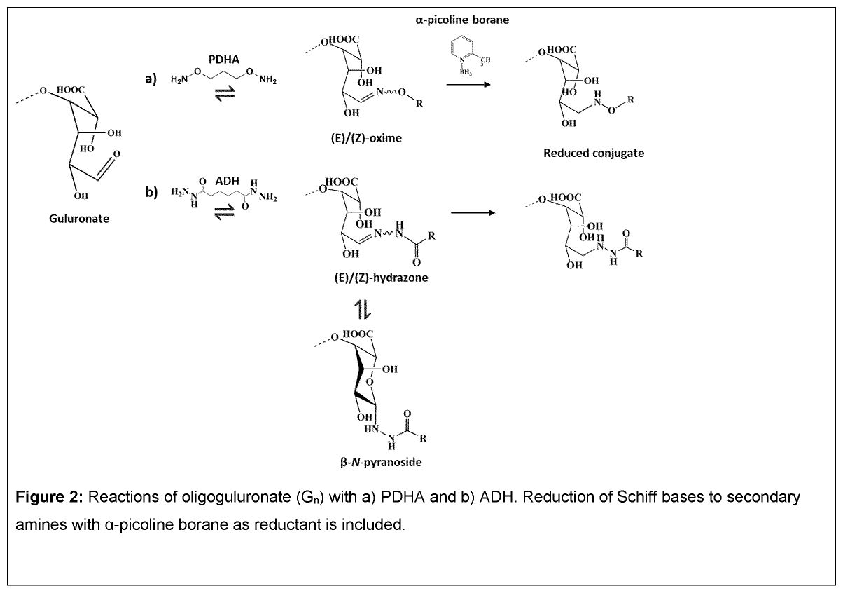

For preparative purposes, oligomers were dissolved in NaAc-buffer (500 mM, pH 4) to a final oligomer concentration of 10-20 mM and 10 equivalents PDHA/ADH was added to the reaction. After 24 h, PB (3-20 equiv.) was added to the reaction at room temp. The reaction was left for 24-120 h with stirring. The reaction mixture was subsequently dialyzed (if DPn<7 with 100-500 Da MWCO and if DPn≥7 with 3.5 kDa MWCO) first against 50 mM NaCl, then against MQ water. Excess linker was removed by semi-preparative SEC, after which samples were dialyzed and freeze-dried. FIG. 2 depicts reactions which occur. These conjugates can be combined with the second polymer.

### Comparative Preparation of Guluronate Diblocks

Guluronate was dissolved in 500 mM Na-Ac buffer (500 mM, pH 4) to a final concentration of 20 mM. 0.5 equivalents and 6-20 equivalents PB was added. Reaction times of 24 h was used for ADH and 120 h for PDHA. The reaction mixture was purified by GFC, dialysis and freeze drying. The guluronate diblock, when exposed to calcium ions, formed a precipitate.

### Inventive Preparation of Guluronate-Linker-dextran Block Copolymers—General Protocol

Dextran was activated with 10 equiv. PDHA and purified. Guluronate (2-3 equiv.) and Dextran-PDHA was dissolved in NaAc-buffer, after 24 h PB was added (3-10 equivalents), and the reaction was left on magnetic stirring for 120 h. The reaction mixture was subsequently dialyzed and freeze dried before purification by semi-preparative GFC, dialysis and freeze drying.

### Particle Formation of G-Linker-Dextran

Gₙ-Linker-Dexₘ (n=12 and m=100) (5-10 mg/ml) was dissolved in 1 ml 10 mM NaCl and filtered (0.22 μm). After 24 h, the sample was dialyzed (Float-A-Lyzer 100-500 Da) against 20 mM CaCl₂ with 10 mM NaCl (1-1.5 L).

The Mn, Mw, and DPₙ from SEC MALS analyses of Dexₘ-b-Gₙ block copolymer (after purification by SEC) and the starting material (Gₙ and Dexₘ-Linker) is presented in table 1.

TABLE 1

| Sample | Mₙ (kDa) | M_{w} (kDa) | DPₙ |
|---|---|---|---|
| Gₙ | 2.5 | 2.5 | 12 |
| Dexₘ-Linker | 16.2 | 18.3 | 100 |
| Dexₘ-Linker-Gₙ | 18.3 | 20.3 | 112 |

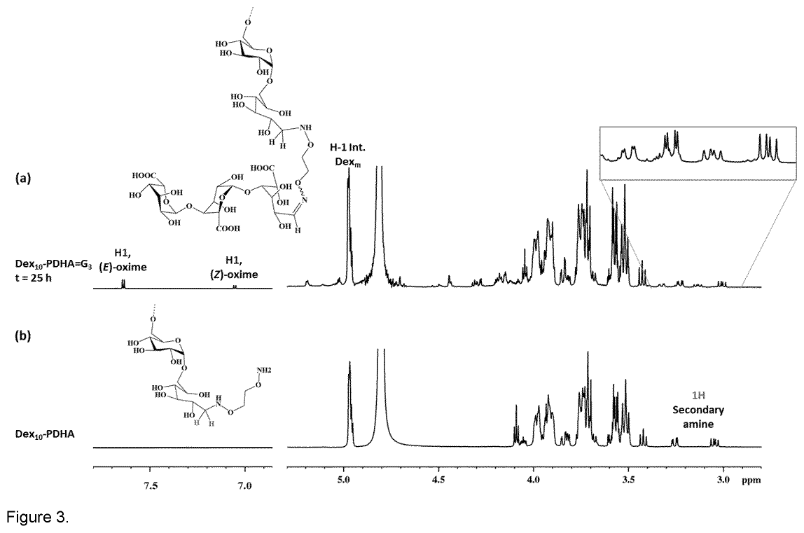

For proof of concept, a further diblock polymer was prepared following the same protocols above and was analysed using NMR. In order to make the NMR easier to assign, shorter chain dextran and guluronic acid oligomers were used. FIG. 3 is the NMR spectra for the Dex₁₀-PDHA-G₃ of the equilibrium reaction mixture with G₃ and PDHA-Dex₁₀ (1:1) in 500 mM AcOHd4 pD 4 (600 MHZ). Resonances from (E)/(Z)-oximes of the conjugate are annotated. The structure of the conjugated G₃=b-Dexₘ is included (=refers to unreduced oxime). 1H-NMR spectra of purified Dex₁₀-PDHA is included for comparison.

In conclusion, the conjugation of oligoguluronate with PDHA-activated dextran chains is efficient for longer and shorter chains, as demonstrated with a DP₁₀₀ dextran chain and a DP₁₀ dextran chain.

### Block Copolymer Self-Assembly in Solution

G₄₀-linker-Dex₁₀₀ diblock polymer in solution was combined with CaCl₂ (20 mM) introduced into the polymer solution by dialysis. A membrane with a cut-off of 100-500 Da was used to minimize the formation of out-of-equilibrium aggregates. After days 10 a steady state had been reached. A population of nanoparticles with diameter around 25 nm corresponds to micellar structures consisting of an alginate-based core hydrogel stabilized by dextran blocks. The hypothesis of a core-shell morphology is supported by the fact that that G₄₀ blocks alone precipitate under similar conditions. Therefore, the diblock structure enabled a strict phase separation between the G-based core and the dextran corona.

### Block Copolymer Self-Assembly in Solution

G₁₁-b-Dex₁₀₀ was prepared analogously. G₁₁-b-Dex₁₀₀ has a markedly different behaviour under similar conditions. Namely, the block copolymer tended to form larger nanoparticles in solution with Ca (1000 nm or more). From a thermodynamic point of view, this could mean that the loss of entropy associated with the formation of a dextran corona is not compensated by a sufficient gain in enthalpy through the gelling of G blocks as they are shorter. Therefore, the ratio of the two blocks length must be carefully considered to have self-assembly properties.

### Further Diblock Polymers

The high reactivity of oligouronates with PDHA implies that reaction with PDHA-activated oligosaccharides to obtain diblock oligo-or polysaccharides would proceed with similar results. This was tested in kinetic studies with β-1,3-glucan-PDHA (DP₉).

In addition, the reaction was also studied with Gₙ-PDHA for preparation of symmetrical blocks. All conjugates (oximes) had been fully reduced with picoline borane (PB) prior to coupling with G₃. These PDHA-activated oligosaccharides represent widely different chemistries (Table 3): dextrans are neutral chains with high chain flexibility due to α-1,6 linkages. Amylose (α-1,4-linked glucans) and β-1,3-glucans are both semi-rigid, neutral chains with the ability to form higher order structures. Collectively they illustrate the versatility of the approach towards almost any type of diblock polysaccharides.

The conjugations with oligoguluronates (Gₙ) were initially studied using a 1:1 molar ratio between the reactants. Results for all PDHA-activated oligosaccharides are summarised in table 2a. Yields were otherwise in the range 40-60%. The preparation of diblock polysaccharides with reduction and purification is further detailed below.

TABLE 2a
Coupling of PDHA-activated oligosaccharides
to oligoguluronates (G₃).

| | Second | | | | Equ. yield (%) |
|---|---|---|---|---|---|
| Activated block | block | Ratio | Conc. B | | prior to oxime |
| (A*) | (B) | (A*-B) | [mM] | pH | reduction |
| G₁₀-PDHA^{#} | G₃ | 1:1 | 7.0 | 4 | 45 |
| Dex₁₀-PDHA | G₃ | 1:1 | 7.0 | 4 | 42 |
| β-1,3-glucan-PDHA | G₃ | 1:1 | 7.0 | 4 | 61 |

^{#}comparative example

The data in table 2 concerns initial experiments using a 1:1 molar ratio between the reactants to obtain reaction kinetics (first order rate constants) and equilibrium yields prior to further oxime reduction.

We subsequently conjugated oligoguluronates (Gₙ) to the activated block using molar ratios in which one or other of the reactants was in molar excess. We generally find that yields are improved where a molar excess of one of the reactants is employed. In particular, the method for diblock preparation and purification might use a molar excess of the activated block relative to the G-block.

For example when the oligoguluronate (7 mM) is reacted with a 3-fold molar excess of PDHA-dextran, reduced, and dialysed, yields are markedly improved. Our research suggests in fact that a 3:1 or 1:3 molar ratio combined with a subsequent reduction step was needed to obtain essentially 100% coupling. If three equivalents of oligoguluronate (relative to PDHA-dextran) were used the diblock could be separated from unreacted oligoguluronate by SEC. Best results and simplest procedures were obtained with three equivalents of PDHA-dextran (relative to oligoguluronate), where the diblock could be selectively precipitated with ethanol while unreacted PDHA-dextran remained in solution and was recycled by standard methods (evaporation/dialysis/freeze-drying).

TABLE 2b
Coupling of PDHA-activated oligosaccharides
to oligoguluronates (G₃).

| | Second | | | | Yield after |
|---|---|---|---|---|---|
| Activated block | block | Ratio | Conc. B | | oxime |
| (A*) | (B) | (A*-B) | [mM] | pH | reduction (%) |
| Dex45-PDHA | G19 | 3:1 | 7.0 | 4 | 100 |

### Purification

After coupling, the diblocks can be purified either by gel filtration chromatography (GFC) or by selective precipitation of unreacted Gₙ (added in excess) with acid. Salt or cooling can be used to further drive the precipitation of excess Gₙ. Noticeably, the conditions should be chosen so that the diblock remains soluble (diblocks short dextran will precipitate more easily compared to one with a higher DPₙ).

When coupling is carried out with an excess of PDHA-dextran, the pure diblock that is formed can be selectively precipitated by adding NaCl to a final concentration of 0.2 M followed by ethanol to 40% (final concentration v/v). The supernatant contains the excess (unreacted) PDHA-dextran, which can be recycled after desalting by dialysis or precipitation with 80% ethanol). There are therefore advantages to the use of excess of the second component both in terms of yield and purification.

### Preparation of Nanoparticles (NPs) by Dialysis or Internal Gelation:

In a further embodiment, nanoparticles can be prepared by dialysis or internal gelation (with CaEGTA or CaCO₃/GDL). The two methods give slightly different particles size and also have different kinetics of assembly.

For these examples a G24-linker-Dex36 diblock polymer was prepared using similar principles to those described above.

### Preparation of NPs by Internal Gelation:

10 mg G₂₄-PDHA-Dex₃₆ was dissolved in 1 ml 15 mM NaCl at 22° C. and placed on shaking for 12 h. 0.3 ml 100 mM CaEGTA was added and the solution was filtered (0.22 μm). 0.0166 g GDL was dissolved in MQ water, filtered and added immediately to the solution with the diblock. The solution was left at 22° C. for 12 h. The formation of nanoparticles was monitored at regular time intervals (every 1-2 h) by dynamic light scattering (DLS) (scattering intensity (kilo counts per second, kcps) and intensity distribution) using ZetaSizer Nano ZS (Malvern Instruments, UK) (25° C., λ=632.8) with back scattering detection (173°).

### Preparation of NPs by Dialysis:

10 mg G₂₄-PDHA-Dex₃₆ was dissolved in 1 ml 10 mM NaCl at 22° C. and placed on shaking for 12 h. The solution was filtered (0.22 μm) and transferred to a dialysis bag. Dialysis against 1 L 20 mM CaCl₂ with 10 mM NaCl was continued for 20 h for MWCO≥3.5 kDa, 14 days for 0.5 kDa<MWCO≤1.0 kDa and 14 days for MWCO≤0.5 kDa. The formation of nanoparticles was monitored by dynamic light scattering (DLS) (scattering intensity (kilo counts per second, kcps) and intensity distribution) using ZetaSizer Nano ZS (Malvern Instruments, UK) (25° C., λ=632.8) with back scattering detection (173°).

Scheme 1 shows the reactions which occur:

### Stability

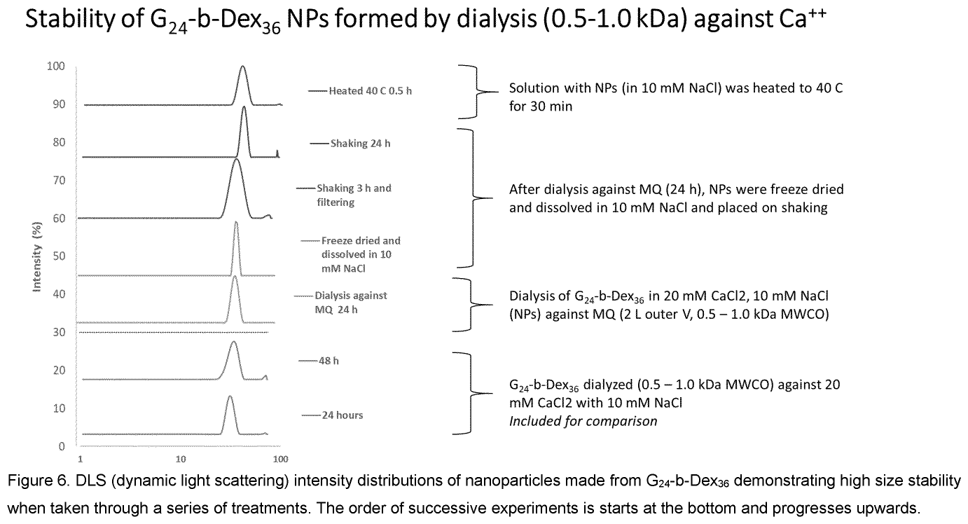

The stability of the nanoparticles for a set of different solvent conditions was demonstrated by dynamic light scattering (DLS). The nanoparticles were shown to be stable upon removal of GDL/EGTA, excess ions (by dialysis against water), and under physiological salt conditions (150 mM NaCl, 1.2 mM CaCl2). The particles could be freeze dried (upon resuspension only a heat treatment (40 C, 30 min) is needed). Results are presented in FIG. 6.

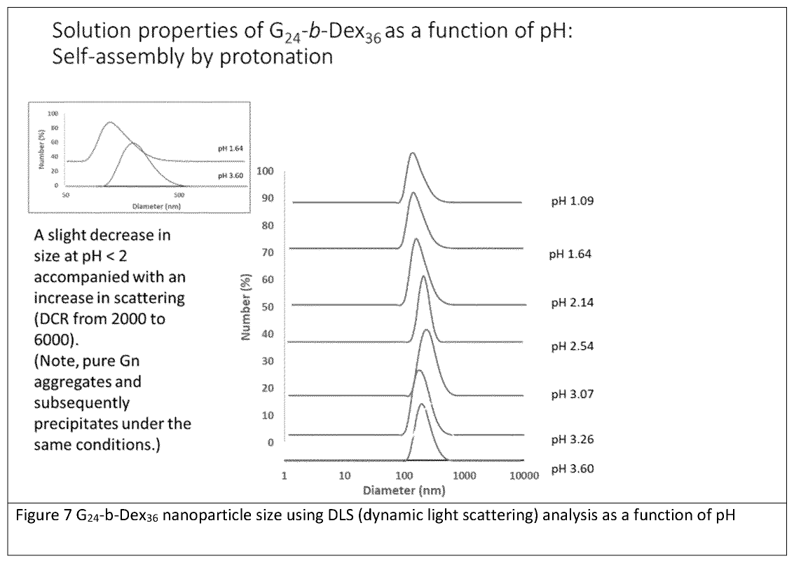

Nanoparticles of G24-b-Dex36 were prepared using acidification. Any residual pure Gn precipitates at low pH, whereas the diblock polymer remains in solution and retains a size corresponding to nanoparticles. The FIG. 7 show this by DLS (dynamic light scattering) analysis presented as number distributions for various pH values down 1.09.

### Nanoparticle Stability in Mg²⁺ Solutions.

A G₄₀-b-Dex₅₀ diblock (4 mg/ml, V=1.0 ml) was dialyzed (float-A-lyzer 3.5-5.0 kDa) against 20 mM CaCl₂ with 10 mM NaCl for 24 h. It was then dialyzed against water (24 h). The process gave NPs and some aggregates with this type of diblock.

The sample was subsequently dialysed for 20-24 h against solutions (20 ml) containing stepwise increasing concentrations of MgCl₂: 0.014 mM, 0.14 mM, 1.4 mM, 14 mM, 140 mM and 1000 mM. The changes in particle size distribution were monitored by DLS. The amounts of Ca²⁺ and Mg²⁺ ions in the dialysate were determined by ICP-MS from which the fractions of bound Ca²⁺ (X_{Ca}) and Mg²⁺ (X_{Mg}) were calculated.

TABLE 3

| | Mg²⁺ dialysis | Fraction of bound Ca²⁺ |
|---|---|---|
| Sample no | solution (mM) | (X_{Ca}) |
| 1 | 0.014 | 1.0 |
| 2 | 0.14 | 0.87 |
| 3 | 1.4 | 0.92 |
| 4 | 14 | 0.77 |
| 5 | 140 | 0.65 |

The results show that the nan remain intact and tend to shrink in size when bound Ca²⁺ is gradually replaced by Mg²⁺ ions. The smallest particles and the narrowest size distributions were obtained for sample 4 (14 mM Mg²⁺, X_{Ca}=0.77). Higher Mg²⁺ concentrations led to particle swelling. Dialysis against appropriate concentrations of Mg²⁺ salts can therefore remove some of the strongly bound Ca²⁺ ions without particle disintegration.

### Diblock Polymer

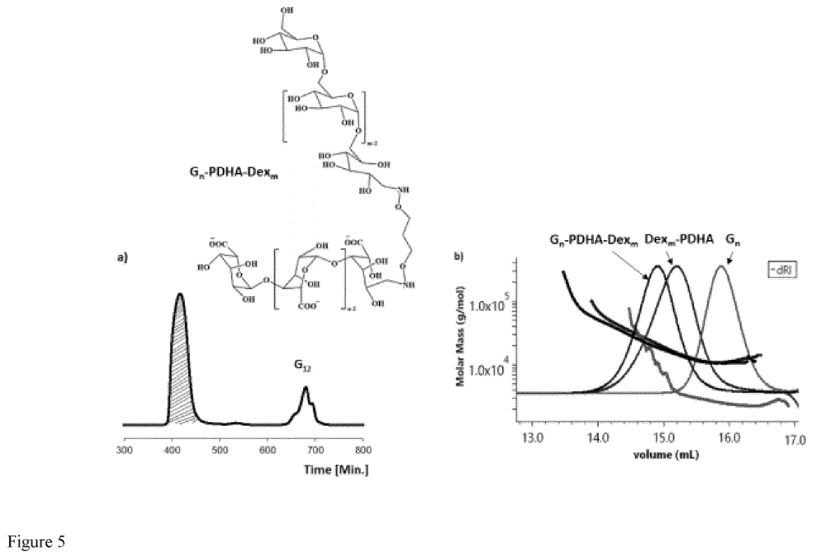

G₁₂-PDHA-Dex₁₀₀ diblock was prepared by reacting free G12 with purified PDHA-dextran with DPn 100. Three equivalents of G12 were here chosen to obtain quantitative substitution of the PDHA-dextran. Residual (unreacted) G12 was selectively removed by SEC (FIG. 5a). SEC-MALLS data for the diblock showed a clear shift in elution profile compared to the free blocks (FIG. 5 b).

### Nanoparticles Containing a Peptide Ligand

A polydisperse G-block with DPₙ=22 was coupled to aminoxy-PEG5 containing a terminal azide group by reductive amination. The Gₙ-aminooxy-PEG-Na was further reacted with cyclooctyne (DBCO) substituted GRGDSP peptide using Cu-free click chemistry to form the Gₙ-aminooxy-PEG-peptide.

The molar mass of the G25-aminooxy-PEG-peptide of 7.9 kDa was determined by SEC-MALLS. The preparation is described in Solberg et al (2022) Carbohydr. Polym. 278, 118840.

Nanoparticles containing 10% (w/w) of G₂₂-aminoxy-PEG-peptide and 90% (w/w) of a G₄₀-b-Dex₅₀ were prepared by the GDL/CaGEGTA method (20 mM CaEGTA, 3.1 equivalents of GDL). The total diblock concentration was 4 mg/ml.

The mixture forms nanoparticles similarly to compositions without the Gₙ-aminooxy-PEG-peptide with only slightly higher hydrodynamic values. No free chains (not incorporated into nanoparticles) could be detected by DLS after adding 0.5 mM BaCl₂, which precipitates free chains. Hence, nanoparticles containing a peptide ligand can be prepared by adding Gₙ-aminoxy-PEG-peptide to a normal Gₙ-b-Dexₘ diblock.

1. A diblock polymer comprising a first component covalently bound via a linker to a second component;
wherein said first component is an oligomer comprising at least 50 mol % L-guluronic acid residues and having a degree of polymerisation n where n is at least 3;
said second component is a polymer having no more than 30 mol % L-guluronic acid residues and having a degree of polymerisation m;
wherein 9n>=m>=n/2.

2. A diblock polymer comprising a first component covalently bound via a linker to a second component;
wherein said first component is an oligomer comprising at least 50 mol % L-guluronic acid residues and having a degree of polymerisation n where n is at least 3;
said second component is an oligo or polysaccharide having no more than 30 mol % L-guluronic acid residues and having a degree of polymerisation m;
wherein 9n=>m=>n/2 and wherein m is 20 or more if n is 20 or less.

3. A diblock polymer comprising a first component covalently bound via a linker to a second component;
wherein said first component is an oligomer comprising at least 50 mol % L-guluronic acid residues;
said second component is a second polymer having no more than 30 mol % L-guluronic acid residues;
wherein said diblock polymer forms a nanoparticle spontaneously in an aqueous solution comprising metal ions in a concentration of at least 0.1 mM of metal ions, such as Ac, Y, Lu, Cu, Ca, Sr, Ba or Ra ions or mixtures thereof, especially Ca, Sr, Ba or Ra ions.

4. A diblock polymer as claimed in any preceding claim wherein the second polymer is an oligo or polysaccharide, poly(meth)acrylate or polyalkylene glycol, especially an oligo or polysaccharide.

5. A diblock polymer as claimed in any preceding claim wherein the second polymer is a dextran or pullulan.

6. A diblock polymer as claimed in any preceding claim wherein the L-guluronic acid oligomer has a degree of polymerisation n of 7 to 70.

7. A diblock polymer as claimed in any preceding claim wherein the second polymer has a degree of polymerisation of 8 to 180.

8. A diblock polymer as claimed in any preceding claim wherein the linker is one that results from an amination, reductive amination or click chemistry.

9. A diblock polymer as claimed in any preceding claim wherein the linker comprises a triazole, two NH—NH—CO— functional groups or two —N—O—CH₂— functional groups.

10. A nanoparticle comprising a diblock polymer as claimed in claims 1 to 9 and positive ions such as metal ions, such as metal 2+ or 3+ ions.

11. A core shell nanoparticle comprising a diblock polymer as claimed in any one of claims 1 to 10, said first component forming the core and said second component forming the shell of said nanoparticle,
wherein metal ions and/or charged organic compounds are ionically bound within the core of the nanoparticle.

12. A nanoparticle as claimed in claim 10 or 11 wherein said metal ions are group (II) metal ions or radionuclides.

13. A nanoparticle as claimed in claims 10 to 12 further comprising a polymer comprising an oligomer comprising at least 50 mol % L-guluronic acid residues and having a degree of polymerisation n where n is at least 3 linked to a biological active molecule such as a peptide.

14. A process for the preparation of a nanoparticle comprising:
(I) obtaining a guluronic acid oligomer such as by hydrolysing alginate in the presence of an acid or base to form a guluronic acid oligomer;
(II) reacting said guluronic acid oligomer with a second polymer carrying a linking group adapted to react with said guluronic acid oligomer to form a diblock polymer.
or
(I) obtaining a guluronic acid oligomer such as by hydrolysing alginate in the presence of an acid or base to form a guluronic acid oligomer;
(II) reacting said guluronic acid oligomer with a linking group adapted to react with said guluronic acid oligomer and with a second polymer;
(III) reacting said guluronic acid oligomer with linking group with a second polymer to form a diblock polymer;
or
(I) obtaining a guluronic acid oligomer, such as by hydrolysing alginate in the presence of an acid or base to form a guluronic acid oligomer, and activating said oligomer with a functional group;
(II) reacting said guluronic acid oligomer with a second polymer which is adapted to carry a functional group that reacts with the functional group of the guluronic acid oligomer to form a diblock polymer;
and subsequently:
contacting said diblock polymer with positive ions, such as metal ions, protons or a charged organic compound to form nanoparticles.

15. A process as claimed in claim 14 wherein the nanoparticles are formed via dialysis or exposure of the nanoparticles to a homogeneous source of metal ions, e.g. a solution of metal ions.

16. A process as claimed in claim 15 wherein exposure of the nanoparticles to a homogeneous source of metal ions involves subjecting an aqueous solution of the diblock polymer and positive ions to a change in pH, preferably using GDL.

17. A process as claimed in claims 14 to 16 wherein said nanoparticles are contacted with a plurality of second metal ions different from those used in the previous step so that said plurality of second metal ions at least partially displace said the metal ions present in said nanoparticles.

18. A process as claimed in claims 15 to 17 further comprising contacting said diblock polymer with positive ions, such as metal ions, protons or a charged organic compound to form nanoparticles in the presence of a polymer comprising an oligomer comprising at least 50 mol % L-guluronic acid residues and having a degree of polymerisation n where n is at least 3 linked to a biological active molecule such as a peptide.

19. Use of a nanoparticle as claimed in claims 10 to 13 to deliver a metal ion or charged organic compound to a patient or to remove a metal ion from a medium containing said metal ion.
